(19) United States
(12) Patent Application Publication
(10) Pub. No.: US 2007/0016018 A1
(43) Pub. Date: Jan. 18, 2007

(54) Review mode graphical user interface for an ultrasound imaging system

(75) Inventor: Rachel M. Kinicki, Acton, MA (US)

Correspondence Address: PHILIPS INTELLECTUAL PROPERTY & STANDARDS, P.O. BOX 3001, BRIARCLIFF MANOR, NY, 10510, US

(73) Assignee: Koninklijke phillips electronics n.v.

(21) Appl. No.: 10/569,251

(22) PCT Filed: Aug. 18, 2004
(86) PCT No.: PCT/IB04/51485

§ 371 Date: Feb. 21, 2006

(51) Int. Cl. A61B 8/00

(52) U.S. Cl. 600/437

(57) ABSTRACT

An improved review mode graphical user interface (GUI) is described in which an acquired ultrasound image can be displayed full-sized, thereby maintaining high image fidelity, while the appropriate review mode controls can still also be displayed, thereby maintaining ease and simplicity of use.

This invention relates to ultrasonic imaging systems and, in particular, to the graphical user interface (GUI) used in review mode on a display device of an ultrasonic imaging system.

There are at least two usage modes of an ultrasound imaging system: the live imaging mode in which the operator of the system is taking one or more ultrasound images of a subject and the review mode in which the operator is reviewing previously acquired ultrasound images, and possibly annotating the acquired ultrasound images. During the live imaging mode, the operator is focused on obtaining an appropriate image (or images) of the area of interest, and the display screen of the ultrasound imaging system will show a live full screen image based on the current input to the ultrasonic transducer so that the operator may manipulate either the ultrasonic transducer or another component in the ultrasound system to obtain a better image. After the operator acquires images during live imaging mode, the operator reviews, analyzes, and annotates the acquired images during review mode, in which the display screen of the ultrasound imaging system shows the acquired images after retrieving them from storage.

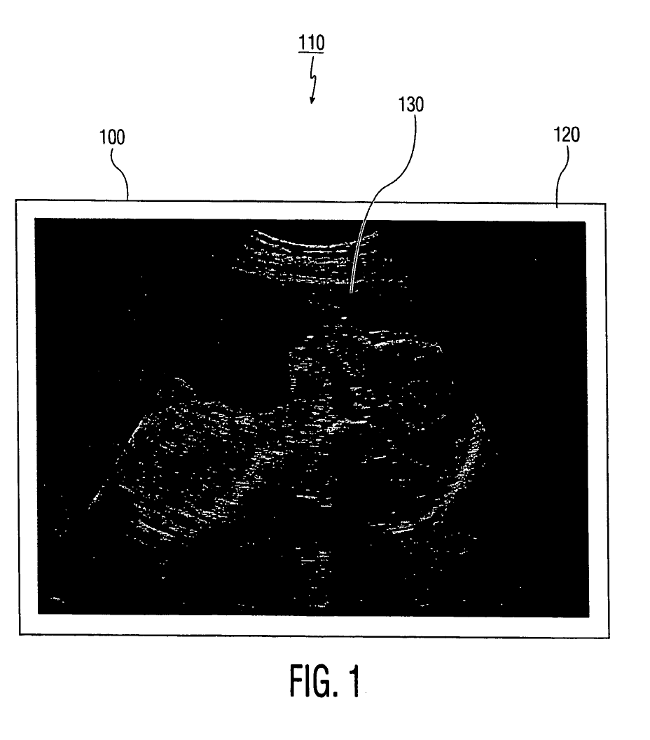

An example of an acquired full screen ultrasound image is shown in FIG. 1, where a display screen 100 displays a full screen ultrasound image 110 of a fetus. Hereinafter, the term “image” may refer to a single image, a group of images, or a time-series sequence of images. Often, a full screen image 110 also has a border 120 around the ultrasound image proper 130. Hereinafter, the phrase “ultrasound image proper” refers to the portion of the full screen image which shows the actual image, e.g., the conic section showing the image of the fetus in FIG. 1. While in live imaging mode, the full screen image 110 also includes data concerning the subject, the area of the body being imaged, the time, the imaging mode being used, and other details concerning the imaging currently being performed. This data usually takes the form of characters and icons which are positioned against the outside edges of the full screen image 110 in order not to obstruct the ultrasound image proper 130. When an image or a continuous series of images are acquired by the ultrasound imaging system, this superimposed data is also acquired and stored.

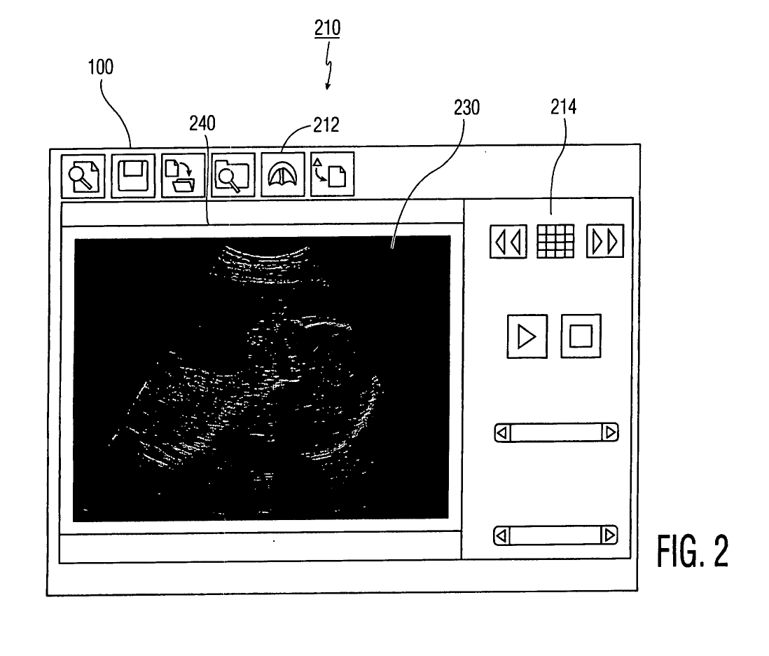

In review mode, the operator needs to manipulate and annotate the ultrasound image proper 130. In order to do so, the operator must have access to various controls, many of which are incorporated into a review mode graphical user interface (GUI) which is shown on the display screen of the ultrasound imaging system. FIG. 2 shows an exemplary review mode GUI 210 displayed on display screen 100. The review mode GUI 210 includes various controls which can be manipulated by the operator through the use of a screen cursor, such as function buttons 212, which are used to initiate various operations (e.g., retrieving an acquired image or series of images, storing an image which has been annotated and modified by the operator during review mode, searching for current studies in the imaging ultrasound system, appending an image to a report, etc.), and image view control buttons 214, which are used to manipulate how the operator views a series of images (e.g., replaying the series of images, stopping the series on a particular frame/image, fast-forwarding through the series, selecting a particular image/frame to view, selecting the speed at which the images are played back, etc.).

However, because the review mode GUI 210 must include these review mode controls, which take up space on display screen 100, the space on display screen 100 available for showing the acquired full screen image is limited to a display window 240, which is much smaller than the full screen of display screen 100. In conventional ultrasound imaging systems, the acquired full screen image is reduced in order to fit within the display window of the review mode GUI. In exemplary FIG. 2, acquired full screen image 110 has been reduced to less than two thirds its size in order to fit within display window 240 of the review mode GUI as reduced image 230.

The decreased size of reduced image 230 necessarily leads to decreased quality of the image itself. Often the subject and imaging data superimposed upon the acquired full screen image 110 becomes so small in size in reduced image 230 as to be unrecognizable. Reduced image quality means that small details in acquired full screen image 110 are lost in reduced image 230, which, in turn, leads to unreliable analysis by the operator during review mode. For example, the operator's ability to correctly measure the size of objects within reduced image 230 is adversely affected by this loss in detail.

In order to fix this problem, other conventional ultrasound imaging systems either superimpose the review mode controls directly on the acquired full screen image or remove the review mode controls completely from the display screen. Yet superimposing the review mode controls onto the acquired full screen image must either risk covering up areas of interest in the acquired full screen image with review mode controls or risk losing functionality by limiting the number of review mode controls available on-screen. Furthermore, removing the review mode controls entirely from the display screen would make it difficult to determine what mode the ultrasound imaging system is in. Having these review mode controls performed via other input means would also make it difficult and counter-intuitive for the user to both manipulate the image and to understand what is being viewed.

Therefore, there is a need for review mode GUI which is capable of showing sufficient detail of the acquired full screen image while not losing the ease and simplicity of on-screen review mode controls.

The present invention provides a review mode GUI for an ultrasound imaging system having a live imaging mode in which ultrasound images of a subject are acquired by said ultrasound imaging system, and a review mode in which acquired ultrasound images are reviewed by a user. When ultrasound images are acquired in live imaging mode, they are large enough to fill the display screen of the ultrasound imaging system. When the acquired full screen ultrasound image is retrieved and displayed in the inventive review mode GUI (obviously during review mode), only a portion of the full-sized retrieved ultrasound image is shown in the display window of the review mode GUI. The display window of the review mode GUI has scroll bars so that the user can change the portion of the acquired full screen ultrasound image displayed in the display window, i.e., the user can view any section of the acquired full screen ultrasound image in full screen size during review mode. Thus, the review mode GUI according to the present invention conserves the original quality and resolution of the acquired full screen ultrasound image, while still retaining the simplicity and ease of GUI controls for manipulating the acquired full screen ultrasound image during review mode.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

In the drawings:

FIG. 1 is the display screen of a typical ultrasound imaging system during live imaging mode showing a full screen image of a subject;

FIG. 2 is the display screen of a prior art ultrasound imaging system displaying a review mode graphical user interface (GUI) being used to review the full screen image acquired during live imaging in FIG. 1;

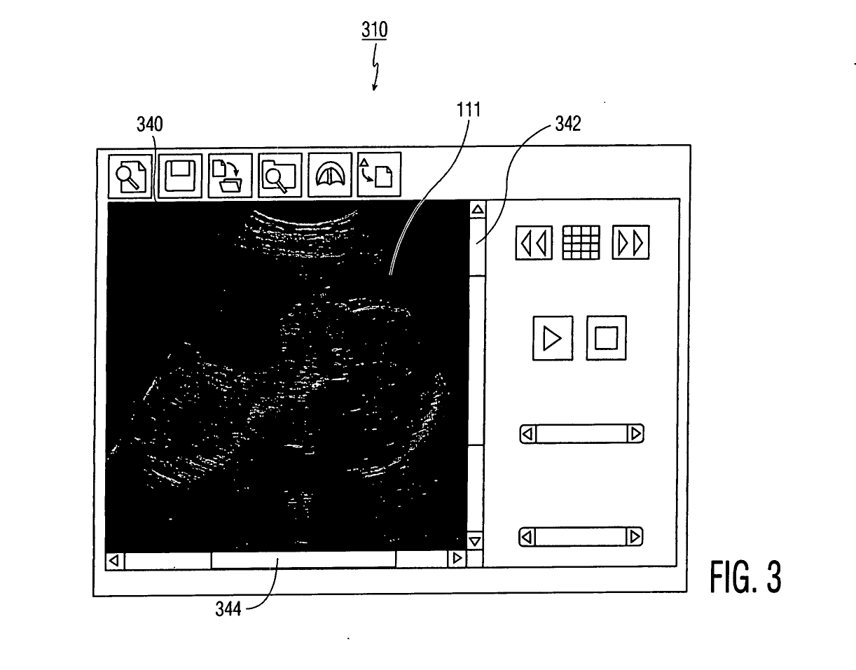

FIG. 3 is the display screen of an ultrasound imaging system displaying a review mode GUI according to a presently preferred embodiment of the present invention, which is being used to review the full screen image acquired during live imaging in FIG. 1, where the display window 340 of review mode GUI 310 has the default view of the central portion 111 of the acquired full screen image 110; and

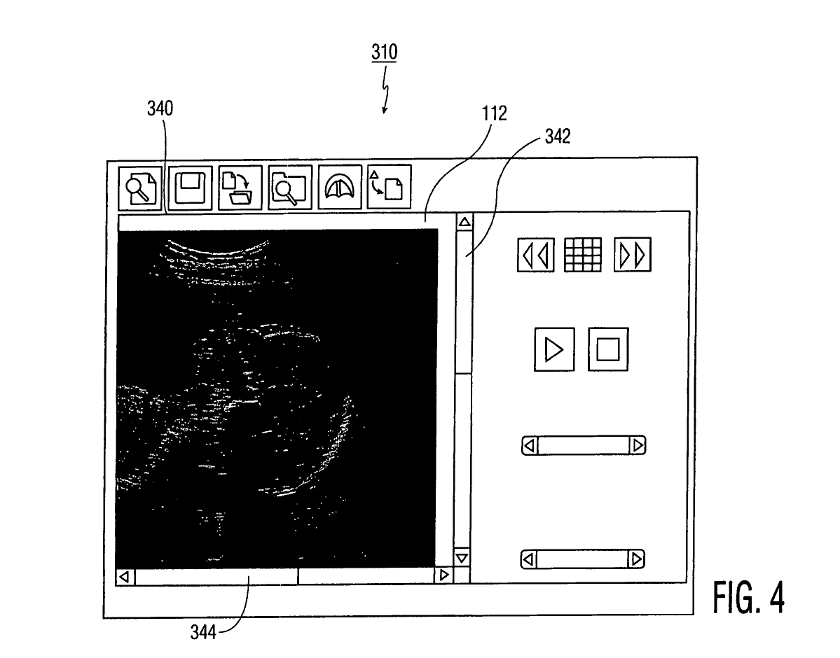

FIG. 4 is the display screen of an ultrasound imaging system displaying a review mode GUI according to a presently preferred embodiment of the present invention, which is being used to review the acquired full screen image from FIG. 1, where the operator has scrolled over using scroll bars 342 and 344 to view the upper right-hand portion 112 of the image in display window 340.

In general, the present invention is directed to a review mode GUI in an ultrasound imaging system which allows both the acquired full screen images to be viewed at their original resolution during review mode, thus not losing details of the imaged subject, and the ease and the intuitive simplicity of on-screen review mode controls. In particular, the present invention provides a review mode GUI which comprises a display window capable of showing the acquired full screen image in its entirety, by a panning function that allows the user to control what portion of the full screen image is shown in the review mode GUI's display window.

A screen shot of a review mode GUI 310 according to the presently preferred embodiment of the present invention is shown in FIG. 3. In FIG. 3, a display window 340 with both a vertical scroll bar 342 and a horizontal scroll bar 344 replaces the prior art display window 240 of the review mode GUI in FIG. 2. Although the size of display window 340 only allows a portion 111 of acquired full screen image 110 to be shown within the review mode GUI 310, the scroll bars 342 and 344 permit the operator to pan across acquired full screen image 110 in order to view any other portion of acquired full screen image 110.

Although the presently preferred embodiment of the review mode GUI shown in FIG. 3 uses scroll bars 342 and 344 to provide a panning function for the user, other means could be used as well according to the present invention. For example, four GUI buttons shaped as arrows pointing in the directions up, down, right, and left could be placed in the review mode GUI, either inside or outside the display window. In such an embodiment, the user would use the cursor to click on one of the arrows, thereby causing the portion displayed to pan over the full screen image in the indicated direction. Furthermore, the cursor itself may provide the means for the user to pan over the full screen image. In such an embodiment, the user could position the cursor on the portion of the full screen image displayed in the display window and click on it to “grab” it. After the image is “grabbed”, the user may move it (thereby panning over the full screen image) in any direction in the same way he or she would have moved the cursor (e.g., by moving the mouse).

According to the presently preferred embodiment of the present invention, the default view of display window 340 is the center of acquired full screen image 110, i.e., when an operator retrieves an acquired full screen image, it is the center portion of the retrieved full screen image which will appear automatically in the display window of the review mode GUI. From the default center position, the operator may pan to any other area of interest in the acquired full screen image.

For example, FIG. 3 shows the default view, with the center portion 111 of acquired full screen image 110 in display window 340. If the operator wishes to view some of the superimposed data (not shown in FIGS.) that often appears in the upper right-hand side of the full screen image, the operator can use scroll bars 342 and 344 to change the view of display window 340 to top right hand portion 112 of acquired full screen image 110, as is shown in FIG. 4. Although data is typically superimposed on the upper right-hand or upper left-hand side of a full screen ultrasound image (above the ultrasound image proper), data may also be placed in the border on the edges of the full screen image, or even on the ultrasound image proper. In any of those cases, review mode GUI 310 according to the presently preferred embodiment of the present invention allows the operator to view those portions of the full screen image at their original size and resolution.

The following example demonstrates some of the advantages of the review mode GUI according to the presently preferred embodiment of the present invention. One use for ultrasound imaging systems is “wall scoring”. An ultrasound image reviewer performs wall scoring when reviewing an ultrasound image of the walls of the heart, by providing a “score” for a section of the cardiac wall which reflects the reviewer's assessment of that section's movement. The numbers for wall scoring range from 0 to 7. A ‘1’ indicates that the section is normal, while the remaining numbers in the range indicate the section is abnormal. When performing wall scoring, the reviewer first selects a section of cardiac wall by means of the review mode GUI and then selects or indicates the appropriate score.

In prior art review mode GUIs, such as GUI 210 in FIG. 2, the reviewer would have to perform wall scoring using a reduced image, such as image 230 in GUI 210. A reduced image degrades wall scoring assessments because the reduction in quality and size of the image diminishes the reviewer's ability either to select an appropriate section or to determine the relative health of that section. By contrast, the presently preferred embodiment of the present invention provides a review mode GUI, such as GUI 310 in FIGS. 3 and 4, which allows the reviewer to view an entire full screen image while performing wall scoring. The panning display window of the inventive review mode GUI provides the reviewer with (1) the ability to review the entire image and search for areas of interest, and (2) the ability to view an area of interest at the original resolution and size of the full screen image. Prior art systems may provide (1) by discarding the review mode GUI, or (2) by shrinking the image to fit within the review mode GUI, but not both (1) and (2).

In conclusion, let us list at least some of the benefits of the present invention over the prior art. The following list is intended to be indicative of the worth of the present invention and is not intended, in any way, shape, or form, to completely define the benefits of the present invention nor to limit the scope of the appended claims directed to the present invention.

First, the review mode GUI provides the operator with a view of the acquired full screen image at its original size and resolution. Because of this, the image quality is not reduced and the operator can make accurate measurements. Second, the present invention retains the advantages of having the review mode controls on the display screen while still providing the operator with access to the full-sized full screen ultrasound image. The review mode GUI also makes it clear the system is currently in review mode, unlike systems which display the acquired full screen image in its full size in review mode. Third, the present invention allows the operator to isolate and focus on areas of interest while temporarily obscuring other sections from view. For example, in the default view, the center portion of the acquired full screen image is shown in the display window, thus allowing the operator to focus on the ultrasound image proper while blocking whatever data may be superimposed on the borders and upper regions of the acquired full screen ultrasound image. When the operator needs such data, the operator may scroll over the full screen image to ascertain it.

Thus, while fundamental novel features of the invention as applied to a preferred embodiment thereof have been shown and described and pointed out, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for providing a review mode graphical user interface (GUI) [310] in a screen [100] of a display device of an ultrasound imaging system, wherein said ultrasound imaging system has at least two modes: a live imaging mode in which ultrasound images of a subject are acquired by the ultrasound imaging system, and a review mode in which at least one acquired ultrasound image is reviewed by a user, wherein said review mode GUI [310] allows the user to view an acquired ultrasound image in full screen size, said method characterized by the steps of:
acquiring and storing an ultrasound image [110] in live imaging mode, said acquired ultrasound image [110] being of such a size that said acquired ultrasound image [110] fills the screen [100] of the display device of the ultrasound imaging system; and
after storing said acquired full screen ultrasound image [110], displaying a portion of the acquired full screen ultrasound image [110] in a display window [340] of the review mode GUI [310], wherein said displayed portion is shown in full screen size within the display window [340];
wherein said display window [340] has a panning function which allows the user to change the portion of the acquired full screen ultrasound image [110] displayed in the display window [340] whereby the user may view any section of the acquired full screen ultrasound image [110] in full screen size during review mode.

2. The method of claim 1, characterized in that said acquired full screen ultrasound image [110] comprises one of a single ultrasound image, a plurality of images, or a time-series sequence of images.

3. The method of claim 1, characterized in that the panning function comprises at least one of at least one scroll bar, at least one arrow button, and at least one cursor function.

4. The method of claim 3, characterized in that the at least one scroll bar comprises a first scroll bar [344] located at the bottom edge of the display window [340] and a second scroll bar [342] located at one of the left edge of the display window [340] or the right edge of the display window [340].

5. The method of claim 3, characterized in that the at least one cursor function comprises a cursor function where, once the user uses a cursor to select the portion of the acquired full screen ultrasound image [110], any movements of the cursor will cause corresponding movements of the acquired full screen ultrasound image [110], thereby changing the portion displayed in the display window [340] of the review mode GUI [310].

6. The method of claim 1, characterized in that the display window [340] of the review mode GUI [310] is of such a size that the displayed portion in the display window [340] comprises most of the acquired full screen ultrasound image [110].

7. The method of claim 1, characterized in that the review mode GUI [310] further comprises function buttons [212] and image view control buttons [214].

8. The method of claim 1, characterized in that, when the displayed portion of the acquired full screen ultrasound image [110] is first presented in the display window [340] of the review mode GUI [310], the displayed portion is a center portion [111] of the acquired full screen ultrasound image [110].

9. The method of claim 1, characterized in that data is superimposed on portions of the acquired full screen ultrasound image [110] during live imaging mode, and said superimposed data is stored with the acquired full screen ultrasound image [110].

10. The method of claim 9, characterized in that the superimposed data comprises information concerning a subject of the ultrasound image [110], an area of the subject which is being imaged, a time and date, an imaging mode being used, and details concerning the imaging being performed during live imaging mode.

11. The method of claim 10, characterized in that the superimposed data takes the form of characters and icons which are positioned against the outside edges of the ultrasound image [110].

12. An ultrasound imaging system having at least two modes: a live imaging mode in which ultrasound images of a subject are acquired by said ultrasound imaging system, and a review mode in which at least one acquired ultrasound image [110] is reviewed by a user, said ultrasound imaging system characterized by:
a display device comprised of at least one display screen [100];
a storage device for storing an ultrasound image [110] acquired in live imaging mode, said acquired ultrasound image [110] being of such a size that said acquired ultrasound image [110] fills the at least one display screen [100] of the display device; and
at least one of hardware, software, and firmware for generating a review mode graphical user interface (GUI) [310] in the at least one display screen [100], said review mode GUI [310] comprising at least a display window [340];
wherein, during review mode, a portion of the acquired full screen ultrasound image [110] is displayed in the display window [340] of the review mode GUI [310], wherein said displayed portion is shown in full screen size within the display window [340]; and
wherein said display window [340] has a panning function which allows the user to change the portion of the acquired full screen ultrasound image [110] displayed in the display window [340] whereby the user may view any section of the acquired full screen ultrasound image [110] in full screen size during review mode.

13. The ultrasound imaging system of claim 12, characterized in that said acquired full screen ultrasound image [110] comprises one of a single ultrasound image, a plurality of images, or a time-series sequence of images.

14. The ultrasound imaging system of claim 12, characterized in that the panning function comprises at least one of at least one scroll bar, at least one arrow button, and at least one cursor function.

15. The ultrasound imaging system of claim 12, characterized in that the display window [340] of the review mode GUI [310] is of such a size that the displayed portion in the display window [340] comprises most of the acquired full screen ultrasound image [110].

16. The ultrasound imaging system of claim 12, characterized in that, when the displayed portion of the acquired full screen ultrasound image [110] is first presented in the display window [340] of the review mode GUI [310], the displayed portion is a center portion [111] of the acquired full screen ultrasound image [110].

17. A computer-readable storage medium having a program of instructions for execution by a processor connected to, and at least partially controlling, a display device, said program being for generating a review mode graphical user interface (GUI) [310] in said display device, and said review mode GUI [310] being for reviewing an acquired full screen ultrasound image [110], characterized in that said program of instructions comprising instructions for:
providing a review mode GUI [310] comprising at least a display window [340] for displaying an acquired full screen ultrasound image [110];
displaying a portion of the acquired full screen ultrasound image [110] in a display window [340] of the review mode GUI [310], wherein said displayed portion is shown in full screen size within the display window [340]; and
providing a panning function for a user, said panning function comprising at least an ability for the user to change the portion of the acquired full screen ultrasound image [110] being displayed in the display window [340], thus allowing the user to view any section of the acquired full screen ultrasound image [110] in full screen size during review mode.

18. The computer-readable storage medium of claim 17, characterized in that said acquired full screen ultrasound image [110] comprises one of a single ultrasound image, a plurality of images, or a time-series sequence of images.

19. The computer-readable storage medium of claim 17, characterized in that the display window [340] of the review mode GUI [310] is of such a size that the displayed portion in the display window [340] comprises most of the acquired full screen ultrasound image [110].

20. The computer-readable storage medium of claim 17, characterized in that said programming instructions for displaying a portion of the acquired full screen ultrasound image [110] in a display window [340] of the review mode GUI [310] comprises instructions for:
displaying, when the displayed portion of the acquired full screen ultrasound image [110] is first presented in the display window [340] of the review mode GUI [310], a center portion [111] of the acquired full screen ultrasound image [110] as the displayed portion.
